(19) United States
(12) Patent Application Publication
(10) Pub. No.: US 2020/0360349 A1
(43) Pub. Date: Nov. 19, 2020

(54) ADMINISTRATION OF MONOBACTAM FOR THE TREATMENT OF URINARY TRACT INFECTION

(71) Applicant: Novartis AG, Basel (CH)

(72) Inventor: Folkert RECK, Emeryville, CA (US)

(21) Appl. No.: 16/762,204

(22) PCT Filed: Nov. 9, 2018
(86) PCT No.: PCT/EP2018/080764

§ 371 Date: May 7, 2020

Related U.S. Application Data
(60) Provisional application No. 62/584,635, filed on Nov. 10, 2017.

(51) Int. Cl. A61K 31/427; A61P 13/02; A61K 9/00

(52) U.S. Cl. CPC A61K 31/427; A61K 9/0019; A61P 13/02

(57) ABSTRACT

Methods for the treatment of a bacterial infection such as a urinary tract infection are described. In particular, methods for treatment of a bacterial infection, such as a urinary tract infection comprising administration of LYS228 are disclosed.

### BACKGROUND

### Field of the Invention

The present invention provides methods for treating a urinary tract infection, and pharmaceutical formulations and unit dose forms useful in those methods. The invention therefore relates to the fields of medicine and pharmacology.

### Description of Related Art

Urinary tract infections (UTIs) are among the most common infectious diseases. In the U.S., they are responsible for more than seven million physician visits and account for more than 100,000 hospital admissions annually, most often for pyelonephritis. Although the exact prevalence of complicated UTI is not well established, catheter-associated bacteriuria is the most common health-care associated infection worldwide, and at least 40% of all hospital acquired infections are UTIs.

The microbial etiology of UTI has been well established and is reasonably consistent. Enterobacteriaceae remain the predominant pathogens isolated in the urinary tract in patients with UTI. However, the ability to effectively treat infections caused by these pathogens has been compromised by the development of antimicrobial resistance. Antimicrobial resistance is a natural phenomenon in microorganisms, and no single strategy will suffice to contain the emergence and spread of bacteria that become resistant to the available antimicrobial drugs.

Antimicrobial resistance is a complex global public health challenge that threatens the ability to effectively treat infections, including UTIs. It reduces the efficacy of available antibacterial drugs, making the treatment of patients difficult, or results in increased morbidity, prolonged illness, and increased mortality. Patients with infections caused by multi drug resistant Enterobacteriaceae are currently treated with carbapenems (Hooten et al Clin. Infect. Dis. p. 625-63, 2010, Golan BMC Infect. Dis. p. 313, 2015). Carbapenems are characterized by a broad spectrum of antibacterial activity, however many bacterial strains have acquired the ability to express carbapenemases of the serine-β-lactamase (SBL) and/or metallo-β-lactamase (MBL) classes (i.e. Klebsiella pneumonia carbapenemase and New Delhi metal-β-lactamase-1), rendering carbapenems ineffective. Infections caused by these strains often require the administration of poorly-tolerated antibiotics such as colistin (Pogue et al Clin. Infect. Dis. p. 879-84, 2011, Navarro-San Francisco et al Clin. Microbiol. Infect. p. E72-9, 2013). The US CDC classifies carbapenem-resistant Enterobacteriaceae as an “urgent threat” and Enterobacteriaceae that express extended-spectrum β-lactamases (ESBLs) as a “serious threat” (Antimicrobial resistance threats in the United States, 2013, US Department of Health and Human Services, Centers for Disease Control and Prevention, Available at: https://www.cdc.gov/drugresistance/pdf/ar-threats-2013-508.pdf). Furthermore, the World Health Organization has issued a global priority list of antibiotic-resistant bacteria for drug development of new antibiotics. Enterobacteriaceae, which are a common cause of hospital and community-acquired infections, have been assigned a critical priority status (Global priority list of antibiotic-resistant bacteria to guide research, discovery, and development of new antibiotics (Internet). World Health Organization. Available at: http://www.who.int/medicines/publications/WHO-PPL-Short_Summary_25Feb-ET_NM_WHO.pdf?ua=1).

LYS228 (1-(((Z)-(1-(2-aminothiazol-4-yl)-2-oxo-2-((3S,4R)-2-oxo-4-((2-oxooxazolidin-3-yl)methyl)-1-sulfoazetidin-3-yl)amino)ethylidene)amino)oxy)cyclopropanecarboxylic acid) 1-(((Z)-(1-(2-aminothiazol-4-yl)-2-oxo-2-(((3S,4R)-2-oxo-4-((2-oxooxazolidin-3-yl)methyl)-1-sulfoazetidin-3-yl)amino)ethylidene)amino)oxy)cyclopropanecarboxylic acid:

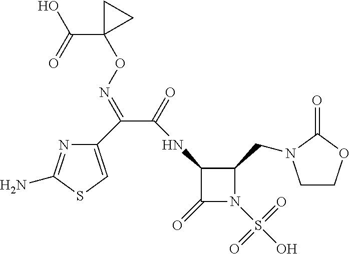 [SMILES: Nc1nc(/C(=N/OC2(C(=O)O)CC2)C(=O)N[C@@H]2C(=O)N(S(=O)(=O)O)[C@@H]2CN2CCOC2=O)cs1]

and including any ionic species thereof is a compound with antibacterial activity (See PCT Application No. PCT/US2015/022011, which is incorporated herein by reference). LYS228 shows strong activity against Gram-negative bacteria, including strains that show resistance to other monobactams. LYS228 kills bacteria by inhibiting cell wall synthesis through covalent modification of the active site serine of penicillin binding protein 3 (PBP3). Aztreonam is the only monobactam approved for clinical use (Tunkel and Scheld Infect Control Hosp Epidemiol p. 486-94, 1990). MBL-expressing Enterobacteriaceae can inactivate all classes of (3-lactam drugs, except monocyclic (3-lactams like the monobactams. MBLs are frequently co-expressed with SBLs. The prevalence of strains expressing the New Delhi metal-β-lactamase-1 (NDM-1) is as high as 12% in clinical isolates from patients with invasive infections in India (Biedenbach et al Antimicrob. Agents Chemother. p. 826-30, 2015, Rahman et al India. Int. J. Antimicrob. Agents p. 30-7, 2014); infections caused by strains expressing NDM-1 and other MBLs have been detected globally, including in Europe, the United States, China, and Japan (Kazmierczak et al Antimicrob. Agents Chemother. p. 1067-78, 2016). Further, the prevalence of MBL-expressing Enterobacteriaceae is increasing in environmental isolates and is expected to increase rapidly in the clinic as they are likely to be selected following the recent introduction of novel —-lactam antibiotics such as ceftazidime/avibactam that cover strains expressing SBLs but not MBLs. The non-clinical experience with LYS228, including safety pharmacology and repeat dose toxicity studies, as well as study in healthy volunteers, supports initial trials in patients to determine its pharmacokinetics, tolerability and efficacy. The LYS228 safety profile appears consistent with the clinical safety observed for other 13-lactam antibiotics presently marketed or those under clinical evaluation for which safety data have been disclosed.

There is a need in the art for improved methods for treating patients with infections caused by Enterobacteriaceae, including those expressing ESBLs and carbapenemases. The present invention addresses these needs and provides for methods of treating bacterial infections.

### SUMMARY

The present invention relates generally to methods for the treatment of a urinary tract infection. In particular, the invention relates to administration of LYS228. These and other aspects and embodiments of the present invention are described by the accompanying detailed description.

### DETAILED DESCRIPTION

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“Administering” or “administration of” a drug to a patient (and grammatical equivalents of this phrase) refers to direct administration, which may be administration to a patient by a medical professional or may be self-administration, and/or indirect administration, such as the act of prescribing a drug. For example and without limitation, a physician who instructs a patient to self-administer a drug and/or provides a patient with a prescription for a drug is, for purposes of the present invention, “administering” the drug to the patient.

“Urinary tract infection” refers to a disease characterized by infection of the urinary tract most commonly caused by microorganisms, resulting in pain with urination, frequent urination, and feeling the need to urinate despite having an empty bladder. When it affects the lower urinary tract it can be known as a bladder infection (cystitis) or urethritis and when it affects the upper urinary tract it can be known as a kidney infection (pyelonephritis) or perinephiric disease. A “complicated urinary tract infection” (cUTI) is a urinary infection occurring in a patient with a metabolic, structural or functional abnormality of the genitourinary tract. Metabolic abnormalities, include but are not limited to diabetes, pregnancy, etc. Structural abnormalities, include but are not limited to calculi, infected cysts, renal/bladder abscesses, certain forms of pyelonephritis, spinal cord injury (SCI), catheters, and the like. Urinary tract infections can also be classified as acute or non-acute.

A “patient” or “subject” refers to a mammal in need of treatment for urinary tract infection. Generally, the patient is a human. In other embodiments of the invention, however, the patient is a non-human mammal, such as a non-human primate, a dog, cat, cow, horse, rabbit, pig, or the like.

“Treatment” or “therapy” refers to a method for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delaying or slowing of disease progression, amelioration or palliation of the disease state, and remission (whether partial or total). “Treatment” can also mean prolonging survival as compared to expected survival in the absence of receiving treatment.

The following enumerated embodiments of the invention are representative:
1. A pharmaceutical composition comprising an effective dose of LYS228, (1-(((Z)-(1-(2-aminothiazol-4-yl)-2-oxo-2-(((3S,4R)-2-oxo-4-((2-oxooxazolidin-3-yl)methyl)-1-sulfoazetidin-3-yl)amino)ethylidene)amino)oxy)cyclopropanecarboxylic acid) 1-(((Z)-(1-(2-aminothiazol-4-yl)-2-oxo-2-((3S,4R)-2-oxo-4-((2-oxooxazolidin-3-yl)methyl)-1-sulfoazetidin-3-yl)amino)ethylidene)amino)oxy)cyclopropanecarboxylic acid):

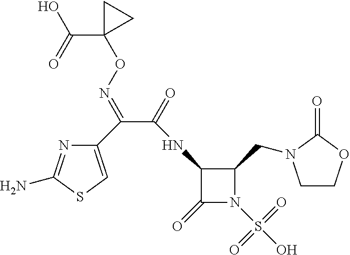 [SMILES: Nc1nc(/C(=N/OC2(C(=O)O)CC2)C(=O)N[C@@H]2C(=O)N(S(=O)(=O)O)[C@@H]2CN2CCOC2=O)cs1]

and including any ionic species thereof formulated for infusion.
2. The pharmaceutical composition of any of the embodiments disclosed herein, wherein the pharmaceutical composition includes instructions for use of the composition in treating a urinary tract infection.
3. The pharmaceutical composition of any of the embodiments disclosed herein, wherein a single intravenous infusion dose of the LYS228 is about 300 mg to about 1000 mg to about 2000 mg over about up to 1 hour to about 3000 mg over about 1 to about 2 hours to about 6000 mg over about 3 or greater hours administered from about every 3 to about 4 to about 5 to about 6 hours.
4. A method for treating a urinary tract infection, said method comprising administering to a patient in need of treatment for a urinary tract infection a therapeutically effective dose of LYS228.
5. The method of any of the embodiments disclosed herein wherein LYS228 is administered in an amount of about 300 mg to about 6000 mg per infusion in a single or divided doses.
6. The method of any of the embodiments disclosed herein, wherein said urinary tract infection is resistant to a carbapenem or f3-lactam antibiotic.
7. The method of any of the embodiments disclosed herein further comprising the steps of:
(i) determining whether the patient has mild or
moderate renal dysfunction, and (ii) administering a lower dose of LYS228 as shown in Table 1 to said patient with mild or moderate renal dysfunction.
8. The method of treating a urinary tract infection of any of the embodiments disclosed herein said method comprising administering to a patient in need of such treatment a therapeutically effective amount of LYS 228, wherein said patient is identified as having a urinary tract infection resistant to resistant to a carbapenem or 13-lactam antibiotic prior to administration of LYS228.
9. The method of of any of the embodiments disclosed herein, wherein the LYS228 is administered for one or more 5 to 14 day cycles, each cycle comprising administering LYS228 at least once daily.

### Administration Regimens

It will be appreciated that therapy sometimes involves multiple “rounds” or “dosage cycles” of administration of a drug, where each cycle comprises administration of the drug one or more times according to a specified schedule. A cycle is generally (but not necessarily) measured in days and can be, for example, 5 to 14 days in duration. In some embodiments, a cycle is longer than 14 days. For example, drugs can be administered for from 1 or more dosage cycles. In a dosage cycle, a drug is administered according to a specified schedule e.g., daily; multiple times a week on consecutive days; etc. When more than one drug (e.g., two drugs) is administered to a subject, each can be administered according to its own schedule as illustrated herein (e.g., daily; etc.). It will be clear that administration of drugs, even those administered with different periodicity, can be coordinated so that both drugs are administered on the same day at least some of the time.

Two drugs are administered to a subject “in combination” when the drugs are administered as part of the same course of therapy. A course of therapy refers to administration of combinations of drugs believed by the medical professional to work together additively, complementarily, synergistically, or otherwise to produce a more favorable outcome than that anticipated for administration of a single drug.

When two drugs are administered in combination, a variety of schedules can be used. In one case, for example and without limitation, Drug 1 is first administered prior to administration of Drug 2, and treatment with Drug 1 is continued throughout the course of administration of Drug 2; alternatively Drug 1 is administered after the initiation or completion of Drug 2 therapy; alternatively, Drug 1 is first administered contemporaneously with the initiation of the other therapy. As used in this context, “contemporaneously” means the two drugs are administered the same day, or on consecutive days.

Although in principle certain drugs can be co-formulated, in general they are administered in separate compositions. Similarly, although certain drugs can be administered simultaneously, more often (especially for drugs administered by infusion) drugs are administered at different times on the same day, on consecutive days, or according to another schedule.

In some embodiments of the invention LYS228 may be administered as a “single agent,” i.e., not administered “in combination” with another antibacterial drug.

The present invention having been described in detail in the preceding sections, the following examples are provided to illustrate certain aspects of, but not to limit, the invention.

### EXAMPLES

### Example 1

LYS228 for the Treatment of cUTI

The following example is provided to illustrate treatment of cUTI with LYS228. A clinical study is conducted to evaluate safety, pharmacokinetics, clinical response, safety and tolerability of LYS228 in patients with urinary tract infection (UTI).

Subjects are divided into two groups: (i) subjects who will be treated with LYS228 (Group I); and (ii) subjects who will be treated with a standard of care antibiotic therapy for the treatment of cUTI (Group II). The subjects are assigned to cohorts. All patients will receive intravenous LYS228 or standard of care therapy in an in-patient setting for a minimum of 5 days and up to 14 days.

LYS228 is safe and well-tolerated following single doses up to 6000 mg. The following doses (Table 1) are designed to achieve >90% probability of target attainment of the percentage of time that the free plasma LYS228 concentration is above the MIC during the dosing interval (fT %>MIC) of 65% with an MIC of up to 2 μg/mL and fT %>MIC of 50% with an MIC of up to 4 μg/mL.

TABLE 1
LYS228 dose based on renal function

| | | | |
|---|---|---|---|
| Creatinine clearance | | Dosing | Infusion |
| (CrCL) | Dose | interval | duration |

| | | | | |
|---|---|---|---|---|
| ≥90 | mL/min | 3 grams | 6 hours | 2 hours |
| 30-89 | mL/min | 2 grams | 6 hours | 2 hours |

LYS228 is administered every day of a 5 to 14 day cycle. LYS228 (300, 1000, 2000, 3000 and 6000 mg) is administered intravenously over about 1 to about 3 hours once every 4 to 6 hours each day of each 5 to 14 day cycle. The treatment is optionally extended for additional 5 to 14 day cycles.

Vital signs, electrocardiograms, clinical laboratory test results, and adverse events are used to assess safety. Clinical evaluation of efficacy, including determined by Common Terminology Criteria for Adverse Events (CTCAE) grading criteria, are performed at baseline and every day while subjects are on the study. Pharmacokinetic parameters are determined from plasma concentrations of LYS228 obtained at specific time intervals after dosing. Blood samples are analyzed for LYS228 levels. All statistical tests used for the analysis of efficacy and safety data are two-sided and performed at 80% confidence interval is computed.

Cmax, Tmax, AUCTau, T1/2, %fT>MIC, CL and Vss will be assessed from the plasma concentration-time data and will be computed for each subject. Efficacy outcomes are evaluated as determined by clinical response to LYS228 compared to standard of care antibiotics for treating patients with cUTI.

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

1. A pharmaceutical composition comprising an effective dose of LYS228, (1-(((Z)-(1-(2-aminothiazol-4-yl)-2-oxo-2-(((3S,4R)-2-oxo-4-((2-oxooxazolidin-3-yl) methyl)-1-sulfoazetidin-3-yl)amino)ethylidene)amino)oxy)cyclopropanecarboxylic acid) 1-(((Z)-(1-(2-aminothiazol-4-yl)-2-oxo-2-(((3S,4R)-2-oxo-4-((2-oxooxazolidin-3-yl)methyl)-1-sulfoazetidin-3-yl)amino)ethylidene)amino)oxy)cyclopropanecarboxylic acid): 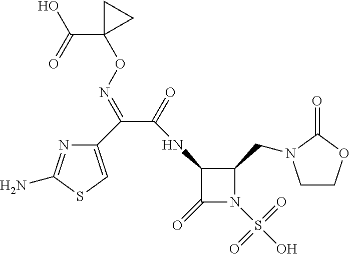 [SMILES: Nc1nc(/C(=N/OC2(C(=O)O)CC2)C(=O)N[C@@H]2C(=O)N(S(=O)(=O)O)[C@@H]2CN2CCOC2=O)cs1]
and including any ionic species thereof formulated for infusion.

2. (canceled)

3. The pharmaceutical composition of claim 1, wherein the pharmaceutical composition includes instructions for use in treating a urinary tract infection.

4. (canceled)

5. The pharmaceutical composition of claim 1, wherein the effective dose is a single intravenous infusion dose of the LYS228 is about 300 mg to 6000 mg over about 1 to about 3 or more hours.

6. A method for treating a urinary tract infection, said method comprising administering to a patient in need of treatment for a urinary tract infection a therapeutically effective dose of LYS228.

7. The method of claim 6 wherein LYS228 is administered in an amount of about 300 mg to about 6000 mg per infusion in a single or a divided dose.

8. The method of claim 6, wherein said urinary tract infection is resistant to a carbapenem or β-lactam antibiotic.

9. The method of claim 6 further comprising the steps of: (i) determining whether the patient has mild or moderate renal dysfunction, and (ii) administering a lower dose of LYS228 as shown in Table 1 to said patient with mild or moderate renal dysfunction.

10. The method of treating a urinary tract infection of claim 6 said method comprising administering to a patient in need of such treatment a therapeutically effective amount of LYS 228, wherein said patient is identified as having a urinary tract infection resistant to resistant to a carbapenem or &-lactam antibiotic prior to administration of LYS228.

11. The method of claim 6, wherein the LYS228 is administered for one or more 5 to 14 day cycles, each cycle comprising administering LYS228 at least once daily.

12. The pharmaceutical composition of claim 5, wherein the single intravenous infusion dose of the LYS228 is adapted for administration in an amount of about 300 mg to about 3000 mg over about 1 to about 2 hours.

13. The pharmaceutical composition of claim 12, wherein the single intravenous infusion dose of the LYS228 is adapted for administration in an amount of about 300 mg to about 2000 mg over about 1 hour.

14. The pharmaceutical composition of claim 13, wherein the single intravenous infusion dose of the LYS228 is adapted for administration in an amount of about 300 mg to about 1000 mg over about 1 hour.

15. The method of claim 7, wherein the dose is administered from about every 3 to about every 6 hours.
